(19) United States
(12) Patent Application Publication
(10) Pub. No.: US 2025/0189533 A1
(43) Pub. Date: Jun. 12, 2025

(54) BEAD-BASED ASSAY TO SCREEN FOR INHIBITOR PROTEIN-GLYCAN INTERACTIONS IN NEURODEGENERATION

(71) Applicant: Rensselaer Polytechnic Institute, Troy, NY (US)

(72) Inventors: Shannon Faris, Westport, MA (US); Chunyu Wang, Latham, NY (US)

(73) Assignee: Rensselaer Polytechnic Institute, Troy, NY (US)

(21) Appl. No.: 18/977,315

(22) Filed: Dec. 11, 2024

Related U.S. Application Data
(60) Provisional application No. 63/730,167, filed on Dec. 10, 2024.
(60) Provisional application No. 63/608,451, filed on Dec. 11, 2023.

(51) Int. Cl. G01N 33/68; G01N 33/542

(52) U.S. Cl. CPC G01N 33/6845; G01N 33/542; G01N 33/6896; G01N 2500/02

(57) ABSTRACT

A bead-based assay system and methods for screening molecule inhibitors of protein-glycan interactions. The assay system includes at least one donor bead and at least one acceptor bead. A first molecule is coupled to the at least one donor bead. The first molecule comprises a protein. A second molecule is coupled to the at least one acceptor bead. The second molecule comprises a glycan. The bead-based assay system includes a screening molecule to be screened for inhibiting protein-glycan interactions. The screening molecule is enabled to interact at a protein-glycan interface of the first molecule and the second molecule.

### CROSS-REFERENCE TO RELATED APPLICATIONS

This application claims the benefit of U.S. Provisional Patent Application No. 63/608,451, filed Dec. 11, 2023, and U.S. Provisional Patent Application No. 63/730,167, filed Dec. 10, 2024, which are incorporated by reference as if disclosed herein in their entireties.

### STATEMENT REGARDING FEDERALLY SPONSORED RESEARCH AND DEVELOPMENT

This invention was made with U.S. Government support under Grant Numbers AG057464-01 and AG069039, awarded by the National Institutes of Health. The United States Government has certain rights in this invention.

### BACKGROUND

Tauopathies, including Alzheimer's disease and frontotemporal dementia, are progressive neurodegenerative diseases characterized by the intracellular deposition of misfolded and aggregated microtubule-associated protein tau fibrils in the brain. Under normal physiological conditions, intrinsically disordered tau binds to microtubules and promotes their stability. Under pathological conditions, tau aggregates and forms neurofibrillary tangles (NFTs). Misfolded tau can propagate and spread tau pathology in a prion-like manner throughout the brain. In the prion model, tau aggregates are released from a donor cell into the extracellular space, which then bind to the cell surface and are endocytosed into a recipient cell. In the recipient cell, the internalized tau acts as a template to promote the misfolding of endogenous tau, seeding more NFTs. Although the exact mechanism of tau prion-like spread is still not fully understood, it is well established that tau recognition using heparan sulfate (HS) chains on heparan sulfate proteoglycans (HSPGs) on the cell surface is required for tau internalization. Thus, HS and tau interaction is a crucial step in the prion-like spread of tau pathology and can be targeted for novel therapeutics for tauopathies like Alzheimer's disease.

### SUMMARY

Aspects of the following disclosure are directed to embodiments of a bead-based assay system to screen for molecule inhibitors of protein-glycan interactions. The assay system may include at least one donor bead and at least one acceptor bead. A first molecule is coupled to the at least one donor bead, wherein the first molecule comprises a protein. A second molecule coupled to the at least one acceptor bead, wherein the second molecule comprises a glycan. The assay system further includes a screening molecule to be screened for protein-glycan inhibition.

In some embodiments, the assay system includes a detection device. In some embodiments of the bead-based assay system, the detection device is structured to detect a fluorescent emission when the at least one donor bead and the at least one acceptor bead are brought in proximity of one another and the at least one donor bead is excited using a beam of light.

In some embodiments of the bead-based assay, the screening molecule is identified as a molecular inhibitor by a decrease in the detected fluorescence emission as compared to a control assay without the screening molecule. In some embodiments of the bead-based assay, the at least one donor bead is comprised of a nickel compound. In some embodiments of the bead-based assay, the nickel compound is nickel chelate. In some embodiments of the bead-based assay, the at least one acceptor bead is comprised of streptavidin. In some embodiments of the bead-based assay, the first molecule is attached to the at least one donor bead via an affinity tag and the second molecule is attached to the at least one acceptor bead via an affinity tag. In some embodiments of the bead-based assay, the first molecule is His-tagged tau. In some embodiments of the bead-based assay, the second molecule is biotinylated heparin.

Aspects of the following disclosure are directed to embodiments of a method of identifying a compound that disrupt interactions at an interface between a protein and a glycan. The method includes providing a reaction vessel, adding at least one donor bead to the reaction vessel, and adding at least one acceptor bead to the reaction vessel. The method further includes coupling a first molecule with the at least one donor bead, wherein the first molecule comprises the protein. A second molecule is coupled to the at least one acceptor bead, wherein the second molecule comprises the glycan. The compound is added to the reaction vessel and the reaction vessel is shaking to mix and incubated for about 1 hour. The at least one donor bead is excited with a beam of light to cause a light emission from the at least one acceptor bead and the light emission is detected. The light emission is analyzed to determine whether the compound disrupts interactions at the interface between the first molecule and the second molecule.

In some embodiments of the method, the beam of light comprises a wavelength of 680 nm. In some embodiments, the method further includes sealing the reaction vessel prior to the shaking. In some embodiments of the method, one or more steps are performed in darkness. In some embodiments, the incubation is performed in darkness. In some embodiments, the method, further includes comprising the at least donor bead of a nickel compound. In some embodiments of the method, the nickel compound comprises nickel chelate. In some embodiments, the method, further includes comprising the at least one acceptor bead of streptavidin. In some embodiments of the method, the first molecule is His-tagged tau. In some embodiments of the method, the second molecule is biotinylated heparin. In some embodiments the method further includes identifying the compound as a molecular inhibitor by a decrease in the detected fluorescence emission as compared to a control assay without the compound. In some embodiments of the method, the reaction vessel comprises a microplate including a plurality of wells. In some embodiments, the method further includes adding different concentrations of the compound to one or more of the plurality of wells.

Aspects of the following disclosure are directed to a composition for treating a neurodegenerative disease. In some embodiments, the composition includes a concentration of one or more active ingredients configured to inhibit protein-glycan interactions. In some embodiments, the one or more active ingredients include 9-hydroxy-2-(2-piperidinylethyl) ellipticinium acetate or a derivative of 9-hydroxy-2-(2-piperidinylethyl) ellipticinium acetate. In some embodiments, the neurodegenerative disease comprises a tauopathy.

Aspects of the following disclosure are directed to a method of treatment that includes administering to a patient an effective amount of a composition including one or more active ingredients configured to inhibit protein-glycan interactions, wherein the one or more active ingredients include 9-hydroxy-2-(2-piperidinylethyl) ellipticinium acetate or a derivative of 9-hydroxy-2-(2-piperidinylethyl) ellipticinium acetate. In some embodiments, the method further includes identifying the patient as having or being suspected of having a neurodegenerative disease. In some embodiments of the method, the neurodegenerative disease comprises a tauopathy.

### BRIEF DESCRIPTION OF THE DRAWINGS

The drawings show embodiments of the disclosed subject matter for the purpose of illustrating the invention. However, it should be understood that the present application is not limited to the precise arrangements and instrumentalities shown in the drawings, wherein:

FIG. 1 schematically illustrates an embodiment of a bead-based assay and assay system according to aspects of the invention;

FIG. 2 schematically illustrates an embodiment of a method of detecting protein-glycan inhibitors using a bead-based assay and assay system according to aspects of the invention;

FIG. 3A shows chemical formulas for six (6) small molecule inhibitors identified using embodiments of the assay system according to aspects of the invention; and

FIG. 3B shows a chemical formula of a small molecule inhibitor, A9, identified as having the greatest protein-glycan inhibition among the small molecules screened using embodiments of the assay system according to aspects of the invention.

### DETAILED DESCRIPTION

The following discussion relates to various embodiments of a bead-based assay system to screen for inhibitor protein-glycan interactions in neurodegeneration and associated methods. It will be understood that the herein described versions are examples that embody certain inventive concepts as detailed herein. To that end, other variations and modifications will be readily apparent to those of sufficient skill. The terms “about” or “approximately” as may be used herein may refer to a range of 80%-120% of the claimed or disclosed value.

HSPGs are composed of HS, a linear glycosaminoglycan (GAG) chain, covalently linked to a core protein. Found virtually on all cell surfaces, HSPGs can bind to numerous proteins through HS. HS-protein binding is driven by electrostatic interactions, between the negative charges of the sulfate groups in HS, and the positively charged amino acids in the protein. A polydisperse structural analog of HS that has been frequently used as an HS mimetic due to its widespread availability is heparin. Heparin differs from HS in that it has a higher sulfation level and a higher content of iduronic acid.

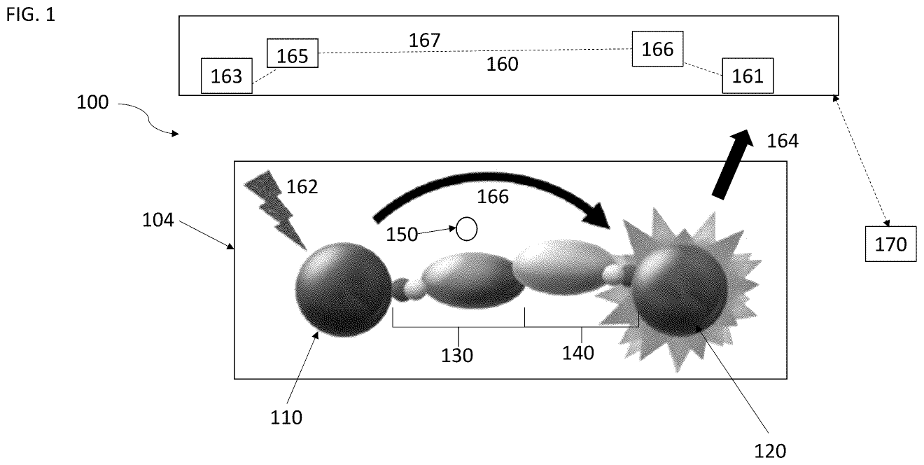

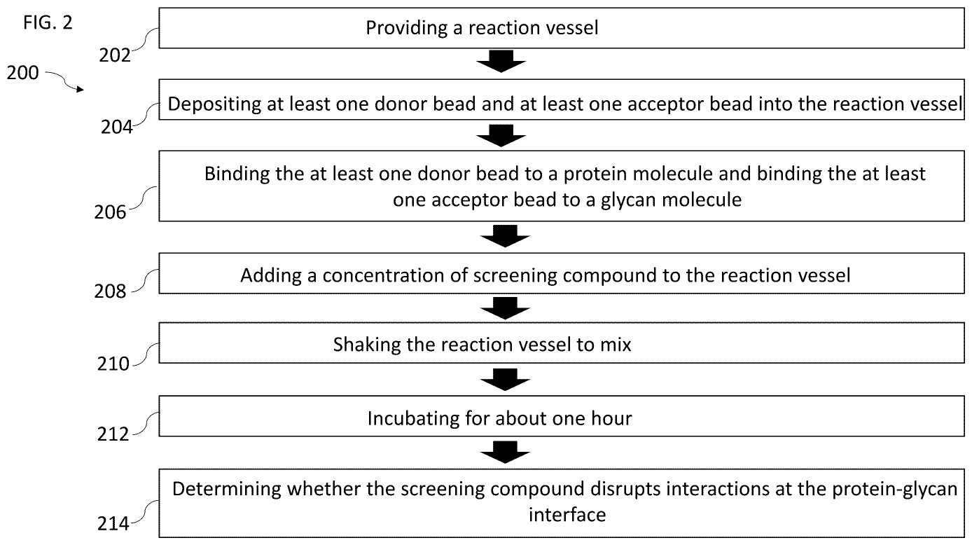

Described herein and referring to FIGS. 1 and 2, embodiments of a bead-based assay system (assay system) to screen for small-molecule inhibitors that disrupt protein-glycan interactions are disclosed. In some embodiments, the assay system comprises an Amplified Luminescent Proximity Homogenous Assay (ALPHA) assay.

In some embodiments, the assay system 100 includes at least one donor bead 110 and at least one acceptor bead 120. In some embodiments, the at least donor bead 110 is comprised of a nickel compound such that the nickel compound is formed as part of the bead 110 or is coated onto the bead 110. In some embodiments, the nickel compound comprises nickel chelate. In some embodiments, the at least one acceptor bead 120 is comprised of a protein such that the protein is incorporated into the bead 120 as the bead 120 is formed or is coated onto the bead 120. In some embodiments, the protein comprises a tetrameric protein, such as streptavidin.

In some embodiments, the first molecule 130 is coupled to or otherwise bound to the at least one donor bead 110. In some embodiments, the first molecule 130 comprises a protein. In some embodiments, the protein is a tubulin associated unit (tau) protein. In some embodiments, the tau protein is modified to include one or more sequences of amino acids at one end. In some embodiments, the amino acids include at least one of the nine (9) essential amino acids, such as histidine (His). In some embodiments, the first molecule 130 is bound to the at least one donor bead 110 via an affinity tag. In some embodiments, the second molecule 140 is coupled to or otherwise bound to the at least on acceptor bead 120. In some embodiments, the second molecule 140 comprises a glycan. In some embodiments, the glycan is heparin, for example biotinylated heparin. In some embodiments, the second molecule 140 is bound to the at least one acceptor bead 120 via an affinity tag. In some embodiments, the first 130 and second molecules 140 are macromolecules.

In some embodiments, the assay system 100 further includes a screening compound or a screening molecule 150 such as a small molecule or small molecule inhibitor. In some embodiments, the screening molecule 150 is a low molecular weight organic compound. In some embodiments, the screening molecule 150 is less than or equal to 1,000 Da. In some embodiments, the screening molecule 150 is less than 900 Da. In some embodiments, the screening molecule 150 is less than 500 Da. In some embodiments, the screening molecule 150 is purified prior to addition to the assay 100.

In some embodiments, the assay system 100 further includes a detection device 160 configured to detect whether the screening molecule inhibits protein-glycan interactions exhibited by the brining the donor 110 and acceptor beads 120 into close proximity of each other. In some embodiments, the detector 160 includes a detecting portion 161, an emitting portion 163, one or more processors 165, memory units 166 and a plurality of electrical circuits 167 between said components. In some embodiments, the detector 160 is in communication with a user interface 170 that is configured to receive user input and output information in response to the user input. In some embodiments, the user interface 170 may be part of the detector 160. In some embodiments, the user interface 170 may include a graphical user interface (GUI), keyboard, mouse, stylus and/or any other device to receive user input and output information in response to the use input.

In some embodiments, the detecting portion 161 is configured to measure a fluorescent emission 164 when the at least one donor bead 110 and the at least one acceptor bead 120 are brought in proximity of one another. In some embodiments, the emitting portion 163 configured to emit a beam of light 162 at a specific wavelength onto the at least one donor bead 110. In some embodiments, the at least one donor bead 110 becomes excited at the specific wavelength, which results in a light emission by the at least one acceptor bead 120. In some embodiments, the beam of light 162 is at a wavelength of 680 nm and causes excitation of the at least one donor bead 110, which results in a singlet oxygen-mediated energy transfer 166 to the at least one acceptor bead 120 and an emission 164 at a wavelength of 620 nm. In some embodiments, the beam of light 162 is generated using a laser and is applied for about 100 ms. In some embodiments, the ability and effectiveness of the screening molecule 150 to inhibit protein-glycan interactions is determined based on the strength of the emission 164, where inhibitors decrease the strength of the emission 164. In other words, the weaker the emission 164 (as compared to a simultaneously run control assay where no screening molecule is provided), the stronger the protein-glycan inhibition of the screening molecule 150. In some embodiments, the user interface outputs information related to the emission 164 detected by the detecting portion 161.

Also referring to FIGS. 1 and 2, the disclosure is also directed to embodiments of a method 200 of screening molecules or screening compounds 150, such as small molecules, to identify those compounds that disrupt interactions at a protein-glycan interface. At 202, in some embodiments, a reaction vessel 104 is provided. The reaction vessel 104 may include any structure that could be used to contain one or more reactants and reagents and enable the one or more reactants and reagents to undergo a chemical reaction. In some embodiments, the reaction vessel 104 may comprise a microplate with a plurality of wells. At 204, at least one donor bead 110 and at least one acceptor bead 120 are deposited into the reaction vessel 104. At 206, the at least one donor bead 110 is bound to, or otherwise captures a first molecule 130 and the at least one acceptor bead 120 is bound to, or otherwise captures a second molecule 140. In some embodiments, the first molecule 120 is a protein and the second molecule 140 is a glycan. In some embodiments, the protein is a tau protein, and the glycan is heparin. In some embodiments, the tau protein includes a histidine tag. In some embodiments, the heparin is biotinylated. In some embodiments, the binding may occur prior to the addition of the at least one donor bead 110 and the at least one acceptor bead 120 to the reaction vessel 104. At 208, the screening compound 150 is added to the reaction vessel 104. In some embodiments, the reaction vessel 104 may include a plurality of wells, which may be used to analyze various concentrations of the screening compound 150 and/or a plurality of different screening compounds 150. In some embodiments, multiple reaction vessels 104 may be used to analyze various concentrations of the screening compound 150 and/or a plurality of different screening compounds 150. At 210, the reaction vessel 104 is shaken to mix its contents and incubated. In some embodiments, the reaction vessel 104 is sealed or otherwise covered before the shaking. In some embodiments, the reaction vessel 104 is shaken for less than a minute. In some embodiments, the reaction vessel is shaken for at least one minute. At 212, the reaction vessel 104 is incubated for at least one hour. In some embodiments, one or more steps are performed in the dark. In some embodiments, the reaction vessel 104 is incubated in the dark. In some embodiments, the reaction vessel is incubated 104 at room temperature (20° C.-25° C.).

At 214, the results are determined, meaning that whether or not the screening compound disrupts interactions at the protein-glycan interface is determined. In some embodiments, in the case of the screening molecule 150 being determined to disrupt interactions at the protein-glycan interface, the relative strength of the disruption may also be determined. In some embodiments, the reaction vessel 104 may be passed through a detector 160 to determine the results of the assay. In some embodiments, the detector 160 exposes the reaction vessel 104 and its contents to a beam of light 162 at a particular wavelength in order to excite the contents of the reaction vessel 104. In some embodiments, the detector 160 generates a beam of light 162 at a wavelength of 680 nm, which causes excitation of the at least one donor bead 110, which results in a singlet oxygen-mediated energy transfer to the at least one acceptor bead 120 and an emission 164 of light at a wavelength of 620 nm. In some embodiments, the strength of the screening molecule 150 to inhibit the protein-glycan interactions is determined based on the strength of the emission 164, where inhibitors decrease the strength of the emission 164. In other words, the weaker the emission (as compared to a simultaneously run control assay where no screening molecule is provided), the stronger the inhibition of the protein-glycan interactions by the screening molecule 150.

In some embodiments, preparation of the tau protein is done according to the procedure described below. In some embodiments, recombinant full-length tau (2N4R, as 1-441) with and without an N-terminal 6×His tag was expressed in E. coli strain BL21-DE3 cells and induced by 0.5 mM isopropyl 13-D-1-thiogalactopyranoside. In some embodiments, the cells were harvested and stored in −20° C. for short-term or −80° C. for long-term storage until purification. In some embodiments, the cell pellets were resuspended in 50 mM Tris, 200 mM NaCl, pH 7.5, supplemented with 1.5× EDTA-free protease inhibitor tablets and 0.1 mM PMSF, then lysed by three passes through a microfluidizer at 80 psi. In some embodiments, the cell debris was pelleted by centrifugation, and the supernatant was boiled in a water bath, chilled on ice for 5 min, and centrifuged. In some embodiments, the supernatant was applied to a 5 mL HisTrap FF column and eluted using an imidazole gradient with final imidazole concentration at 350 mM. In some embodiments, fractions containing tau were pooled and dialyzed in 50 mM Na₂HPO4, 1 mM EDTA, pH 6.5 and applied to a 5 mL SP FF column. In some embodiments, Tau was eluted using NaCl gradient with final concentration of 1 mM. In some embodiments, fractions containing tau were pooled and concentrated, flash-frozen, and stored at −80° C.

### Example

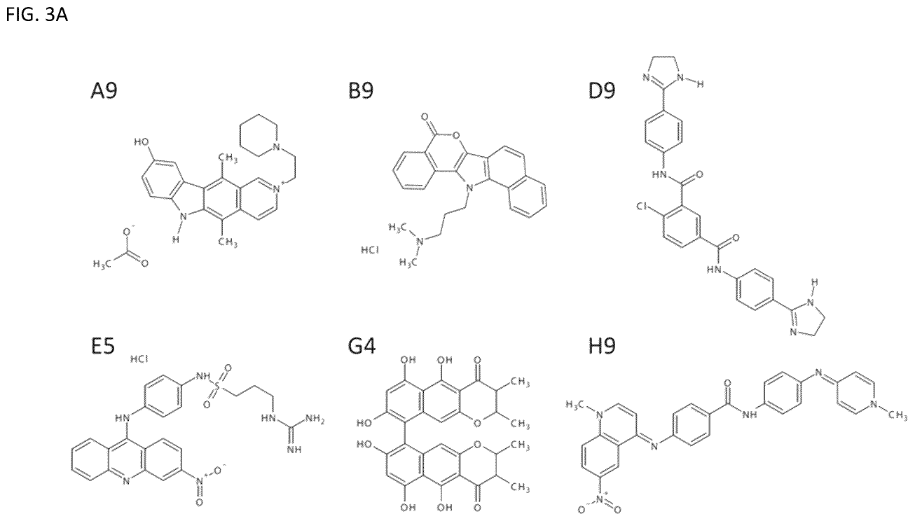

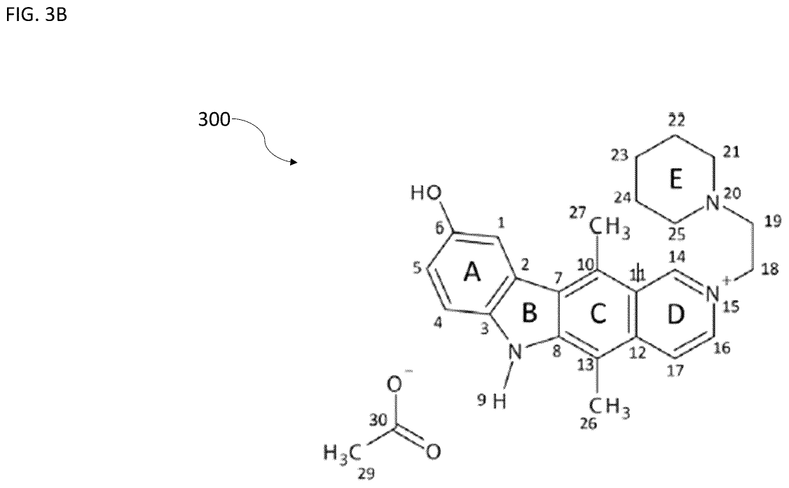

In a particular example, the assays are assembled using OptiPlate-384 flat white bottom plates. In some embodiments, the reactants are diluted in a 1× AlphaLISA HiBlock Buffer master mix (25 mM HEPES, 0.1% casein, 1 mg/mL Dextran-500, 0.5% Triton X-100, 0.5% Blocking reagent, 0.5% BSA and 0.05% Proclin-300, pH 7.4). AlphaLISA Streptavidin Acceptor beads, biotinylated heparin, and 6×His tagged tau (His-tau) were added to the reaction master mix, resulting in final assay concentrations of 5 μg/mL, 0.1 μM, and 0.1 μM respectively. In some embodiments, the addition of the nickel chelate donor beads is done in a dark room to minimize photobleaching due to photosensitivity of the beads, for a final assay concentration of 5 μg/mL. In some embodiments, assays may be assembled in each microplate well by combining a fixed volume of the screening compound at varying concentrations with the master mix. In some embodiments, the microplate may be sealed, shaken gently to mix the components thoroughly, and incubated for 1 h at room temperature in the dark. In some embodiments, laser excitations may be carried out on a Tecan infinite M1000 Pro microplate reader equipped with AlphaScreen Assay software at 680 nm for about 100 ms and with emissions recorded at 620 nm. Each sample was run in triplicate, including two controls, the compound solvent 1% DMSO as a negative (no inhibition) control and 10 μM heparin as a positive (inhibition) control. In an embodiment, six (6) compounds from the NCI diversity Set VI, whose chemical structures are shown in FIG. 3A, were identified with the lowest detected fluorescent signal. These compounds, indicated as A9, B9, D9, E5, G4, and H9, were further probed using embodiments of the disclosed assay system in a dose-dependent manner. Results indicated that A9 exhibited the greatest protein-glycan inhibition of the six (6) compounds.

Referring to FIG. 3B, in some embodiments, the screening compound 150 comprises 9-hydroxy-2-(2-piperidinylethyl) ellipticinium acetate or 6H-Pyrido [4, 9-hydroxy-5,11-dimethyl-2-[2-(1-piperidinyl)ethyl]-, acetate (A9) 300. A9 300 was shown to inhibit heparin-induced tau aggregation in a dose-dependent manner. FIG. 3 shows the number structure of A9 300. A9 300 may comprise a chemical formula of C₂₄H₂₈N₃O·C₂H₃O₂ and a molecular weight of 434.0 g/mol. Additional analysis of A9 300 was done by performing a cell uptake assay utilizing H4 neuroglioma cells. Introduction of A9 300 inhibited tau uptake in a dose-dependent manner. These data suggest that A9 300 may target multiple mechanisms of action (internalization, aggregation) dependent upon the tau-HS interaction. Nuclear magnetic resonance (NMR) was used to probe the tau-A9 interface and A9 300 was found to disrupt the tau-HS interaction by occupying the glycan. Further analysis of A9 300 suggested that A9's aromatic region binds to heparin instead of its aliphatic carbons. In some embodiments, A9 300 is effective to disrupt tau-heparin interaction when present in a higher concentration than the concentration of tau. In some embodiments, A9 300 is effective when present in about a 100-fold molar excess relative to the tau-heparin complex.

The disclosure is also directed to a drug or composition for treating a neurodegenerative disease. In some embodiments, the neurodegenerative disease comprises a tauopathy. In some embodiments, the composition comprises at least one of compounds A9, B9, D9, E5, G4, and H9. In some embodiments, the composition comprises A9. The disclosure is also directed to embodiments of a method of treatment. In some embodiments, the method includes administering an effective amount of a composition to a patient. In some embodiments, the composition includes one or more active ingredients configured to inhibit protein-glycan interactions. In some embodiments, the one or more active ingredients include A9 or a derivative of A9. In some embodiments, the effective amount of the composition of an effective amount of A9 (or a derivative of A9) includes a dose or a concentration of A9 (or a derivative of A9) that produces a biological response. In some embodiments, the biological effect includes a disruption of the protein-glycan (tau-glycan) interaction. In some embodiments, the method further includes identifying the patient as having or being suspected of having a neurodegenerative disease. In some embodiments, the neurodegenerative disease comprises a tauopathy. In some embodiments, a derivative of A9 may be used in place of A9. In some embodiments, a derivative of A9 includes structural changes to A9 that do not substantially affect the protein-glycan interaction inhibition functionality of the structure.

Although the invention has been described and illustrated with respect to exemplary embodiments thereof, it should be understood by those skilled in the art that the foregoing and various other changes, omissions, and additions may be made therein and thereto, without parting from the spirit and scope of the present invention.

What is claimed is:

1. A bead-based assay system to screen for molecule inhibitors of protein-glycan interactions, comprising:
at least one donor bead;
at least one acceptor bead;
a first molecule coupled to the at least one donor bead, wherein the first molecule comprises a protein;
a second molecule coupled to the at least one acceptor bead, wherein the second molecule comprises a glycan; and
a screening molecule to be screened for inhibiting protein-glycan interactions, wherein the screening molecule is enabled to interact at a protein-glycan interface of the first molecule and the second molecule.

2. The bead-based assay system of claim 1, wherein the at least one donor bead is comprised of a nickel compound.

3. The bead-based assay system of claim 2, wherein the nickel compound is nickel chelate.

4. The bead-based assay system of claim 1, wherein the at least one acceptor bead is comprised of streptavidin.

5. The bead-based assay system of claim 1, wherein the first molecule is His-tagged tau.

6. The bead-based assay system of claim 1, wherein the second molecule is biotinylated heparin.

7. The bead-based assay of claim 1, further including a detection device configured to detect a fluorescent emission when the at least one donor bead and the at least one acceptor bead are brought in proximity of one another and the at least one donor bead is excited using a beam of light,
wherein the screening molecule is identified as a molecular inhibitor by a decrease in the detected fluorescence emission as compared to a control bead-based assay without the screening molecule.

8. A method of identifying a compound that disrupts interactions at an interface between a protein and a glycan, comprising:
providing a reaction vessel;
adding at least one donor bead in the reaction vessel;
adding at least one acceptor bead in the reaction vessel;
coupling a first molecule with the at least one donor bead, wherein the first molecule comprises the protein;
coupling a second molecule with the at least one acceptor bead, wherein the second molecule comprises the glycan;
adding the compound to the reaction vessel;
shaking the reaction vessel to mix;
incubating the reaction vessel for about 1 hour;
exciting the at least one donor bead with a beam of light to cause a light emission from the at least one acceptor bead;
detecting the light emission; and
determining whether the compound disrupts interactions at the interface between the first molecule and the second molecule based on the detected light emission.

9. The method of claim 8, wherein the beam of light comprises a wavelength of 680 nm.

10. The method of claim 8, further comprising sealing the reaction vessel prior to shaking.

11. The method of claim 8, wherein the incubation is performed in darkness.

12. The method of claim 8, further including comprising the at least one donor bead of a nickel compound.

13. The method of claim 12, wherein the nickel compound comprises nickel chelate.

14. The method of claim 8, further including comprising the at least one acceptor bead of streptavidin.

15. The method of claim 8, wherein the first molecule is His-tagged tau.

16. The method of claim 8, wherein the second molecule is biotinylated heparin.

17. The method of claim 8, further comprising identifying the compound as a molecular inhibitor by a decrease in the detected fluorescence emission as compared to a control assay without the compound.

18. The method of claim 8, wherein the reaction vessel comprises a microplate including a plurality of wells.

19. The method of claim 18, further comprising adding different concentrations of the compound to one or more of the plurality of wells.

20. A composition for treating a neurodegenerative disease, comprising:
a concentration of one or more active ingredients configured to inhibit protein-glycan interactions,
wherein the one or more active ingredients include 9-hydroxy-2-(2-piperidinylethyl) ellipticinium acetate or a derivative of 9-hydroxy-2-(2-piperidinylethyl) ellipticinium acetate.

21. The drug of claim 20, wherein the neurodegenerative disease comprises a tauopathy.

22. A method of treatment, comprising:
administering to a patient an effective amount of a composition comprising one or more active ingredients configured to inhibit protein-glycan interactions, wherein the one or more active ingredients include 9-hydroxy-2-(2-piperidinylethyl) ellipticinium acetate or a derivative of 9-hydroxy-2-(2-piperidinylethyl) ellipticinium acetate.

23. The method of claim 22, further comprising identifying the patient as having or being suspected of having a neurodegenerative disease.

24. The method of claim 23, wherein the neurodegenerative disease comprises a tauopathy.
